(19) United States
(12) Patent Application Publication
(10) Pub. No.: US 2024/0158161 A1
(43) Pub. Date: May 16, 2024

(54) MEDICAL WASTE COLLECTION DEVICE AND MEDICAL WASTE COLLECTION SYSTEM COMPRISING SAME

(71) Applicant: Hae Shim AN, Busan (KR)

(72) Inventor: Hae Shim AN, Busan (KR)

(21) Appl. No.: 18/281,830

(22) PCT Filed: Oct. 28, 2021
(86) PCT No.: PCT/KR2021/015357

§ 371 Date: Sep. 13, 2023

(30) Foreign Application Priority Data
Jun. 30, 2021 (KR) 10-2021-0085826
Aug. 11, 2021 (KR) 10-2021-0106284

(51) Int. Cl. B65F 1/14; A61L 2/10; B65F 1/06; B65F 1/16; B65F 7/00

(52) U.S. Cl. CPC B65F 1/1405; B65F 1/068; B65F 1/1473; B65F 1/163; B65F 7/00; A61L 2/10; B65F 2210/167; B65F 2210/168

(57) ABSTRACT

A medical waste collection device may include a container part having a first opening at one side and having a bag placed therein, a cover part which is detachably installed on the container part and is for opening and closing the first opening, a compression member which is installed on the cover part and compresses medical waste accommodated in the container part, and a sterilization unit for sterilizing the medical waste accommodated in the container part. The compression member may include a compression plate located in the container part, and a compression rod which passes through the cover part to be connected to the compression plate, and presses the compression plate.

### CROSS REFERENCE TO RELATED APPLICATIONS AND CLAIM OF PRIORITY

This application claims benefit under 35 U.S.C. 119, 120, 121, or 365(c), and is a National Stage entry from International Application No. PCT/KR2021/015357, filed Oct. 28, 2021, which claims priority to the benefit of Korean Patent Application Nos. 10-2021-0085826 filed on Jun. 30, 2021, and 10-2021-0106284 filed on Aug. 11, 2021, in the Korean Intellectual Property Office, the entire contents of which are incorporated herein by reference.

### BACKGROUND

### 1. Technical Field

The present invention relates to a medical waste collection device and a medical waste collection system including the same, and more specifically to, a medical waste collection device for preventing the spread of contact infections and reducing the volume of medical waste and a medical waste collection system including the same.

### 2. Background Art

In general, medical waste refers to various types of waste generated by medical-related institutions such as hospitals, clinics, and public health centers. The medical waste broadly includes not only testing equipment such as cotton pads, gauze, bandages, diapers, human body extracts, dialyzed blood, intravenous fluids, syringes, needles, thermometers, and test tubes, but also analysis devices, X-ray films, waste developers, organic solvents, and the like.

For example, medical waste may be classified into general waste, pathological waste, infectious/potentially infectious waste, damaging waste, chemical waste, medicinal waste, explosive waste, and radioactive waste. Due to its nature, the medical waste has a risk of contamination by pathogenic harmful substances such as pathogens, heavy metals, and poisons, and thus requires special disposal, unlike general household waste. Currently, the medical waste is collected by collection container holders and disposed of by incineration by medical institutions or specialized medical waste disposal companies.

However, in the process of putting the medical waste into the collection container holder and putting the medical waste in the collection container holder into a waste bag, viruses and harmful bacteria could be transmitted to medical workers.

In addition, as the volume of medical waste increases due to the increased use of disposable medical products, the amount of carbon emissions also increases, and harmful gases such as dioxin emitted during the incineration process cause social and environmental problems.

### SUMMARY

The inventive concept of the present invention is to provide a medical waste collection device capable of compressing medical waste to reduce the disposal volume of medical waste, and a medical waste collection system including the same.

Another inventive concept of the present invention is to provide a medical waste collection device capable of sterilizing medical waste contained in a container to minimize infection by bacteria and viruses, and a medical waste collection system including the same.

Another inventive concept of the present invention is to provide a medical waste collection device capable of automatically collecting medical waste to improve convenience, and a medical waste collection system including the same.

The inventive concepts of the present invention are not limited to those mentioned above, and other inventive concepts not mentioned will be clearly understood by a person skilled in the art from the description below.

A medical waste collection device according to the present invention may include a container having a first opening on one side and holding a bag therein; a cover installed in the container and opening and closing the first opening; a compression member installed in the container and compressing the waste contained in the bag; and a sterilization unit that sterilizes the waste contained in the bag; wherein the compression member may include a compression plate located within the container, and a compression rod that passes through the cover, is connected to the compression plate, and presses the compression plate.

A medical waste collection system according to the present invention may include a medical waste collection device; a medical waste pickup device that picks up a bag in a container of the medical waste collection device; and a server that wirelessly communicates with the medical waste collection device and the medical waste pickup device and controls the medical waste collection device and the medical waste pickup device, wherein the medical waste pickup device transmits sealing information to the server through a communication unit when the bag in the container is sealed, and when receiving the sealing information, the server may transmit a movement signal so that the medical waste collection device moves to the medical waste pickup device.

Specific details of other embodiments are included in disclosure and drawings.

According to embodiments of the present invention, the disposal volume of medical waste may be reduced. Accordingly, it is possible to save resources and protect the environment. Additionally, the spread of contact infection may be prevented by allowing a user to open the container and tie the bag without contacting the medical waste collection device. Additionally, the present invention may sterilize medical waste to eliminate infections such as bacteria and viruses.

The effects of the present invention are not limited to the effects mentioned above, and other effects not mentioned will be clearly understood by a person skilled in the art from the description of the claims.

### BRIEF DESCRIPTION OF THE DRAWINGS

FIG. 1 is a schematic cross-sectional view of a medical waste collection device according to an embodiment of the present invention.

FIG. 2 is a schematic cross-sectional view of the medical waste collection device of FIG. 1.

FIG. 3 is a rear view of the medical waste collection device of FIG. 1.

FIG. 4 is a bottom view of the medical waste collection device of FIG. 1.

FIG. 5 is a schematic diagram of a pressing member of FIG. 2.

FIG. 6 is a schematic cross-sectional view of a medical waste collection device according to another embodiment of the present invention.

FIG. 7 is an enlarged view of area A of FIG. 6.

FIG. 8 is a plan view of a gripping unit of FIG. 6.

FIG. 9 is a cross-sectional view taken along line A-A′ in FIG. 8.

FIG. 10 is a cross-sectional view taken along line B-B′ in FIG. 8.

FIG. 11 is a schematic diagram for illustrating a process in which the gripping unit of FIG. 8 grips an upper part of a bag.

FIG. 12 is a schematic cross-sectional view of a medical waste collection device according to another embodiment of the present invention.

FIG. 13 is a schematic diagram of the medical waste collection device of FIG. 12 sealing a bag.

FIG. 14 is a schematic diagram of a medical waste collection system including a medical waste collection device according to an embodiment of the present invention.

### DETAILED DESCRIPTION

The advantages and features of the present invention and a method for achieving those will become clear by referring to the embodiments described in detail below along with the accompanying drawings. However, the present invention is not limited to the embodiments disclosed below, but may be implemented in various different forms. The embodiments are merely provided to ensure that the disclosure of the present invention is complete and to fully inform a person skilled in the art of the scope of the invention to which the present invention pertains, and that the present invention is defined by the scope of the claims. Like reference numerals refer to like elements throughout the specification.

The embodiments described herein will be described with reference to cross-sectional views and/or plan views, which are preferred illustrations of the present invention. In the drawings, the thicknesses of films and areas are exaggerated for effective explanation of technical content. Accordingly, the areas illustrated in the drawings have schematic properties, and the shapes of the areas illustrated in the drawings are intended to illustrate a specific shape of the area of the device and are not intended to limit the scope of the invention. In various embodiments of the present specification, terms such as first, second, and third are used to describe various components, but these components should not be limited by these terms. These terms are merely used to distinguish one component from another. The embodiments described and illustrated herein also include complementary embodiments thereof.

The terms used herein are for illustrative purposes only and are not intended to limit the present invention. As used herein, singular forms also include plural forms, unless specifically stated otherwise in the context. As used herein, “comprises” and/or “comprising” do not exclude the presence or addition of one or more other components, steps, operations and/or devices from the mentioned component, step, operation and/or device.

Unless otherwise defined, all terms (including technical and scientific terms) used in this specification may be used with meanings that can be commonly understood by a person skilled in the art to which the present invention pertains. Additionally, commonly used, predefined terms are not interpreted ideally or excessively unless clearly specifically defined.

Hereinafter, with reference to the drawings, the inventive concept of the present invention and embodiments thereof will be described in detail.

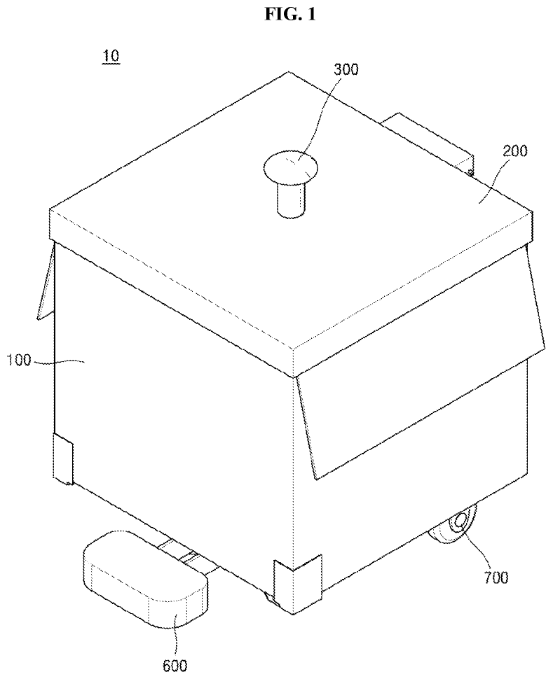

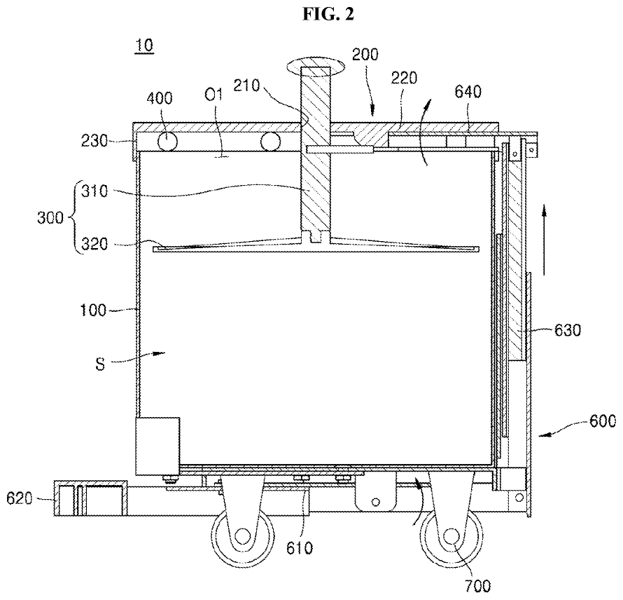

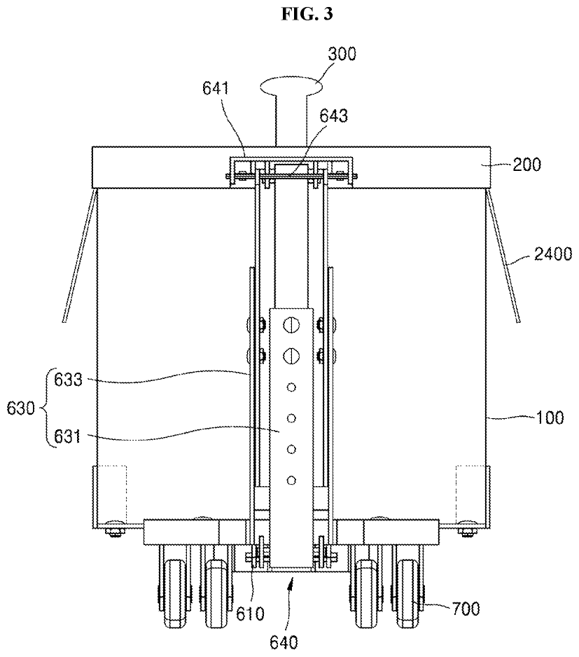

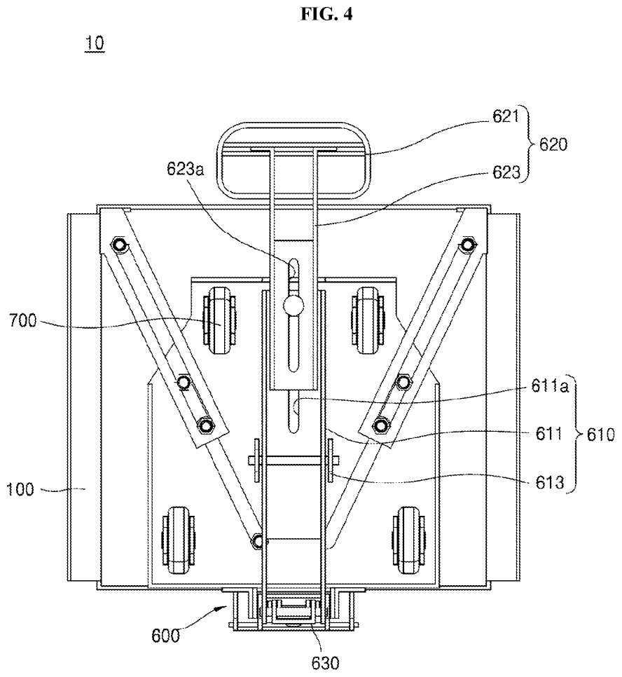

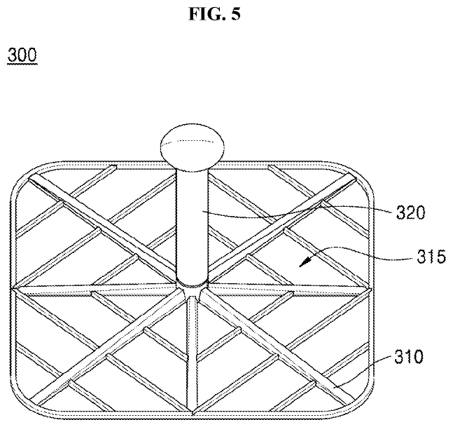

FIG. 1 is a schematic cross-sectional view of a medical waste collection device according to an embodiment of the present invention. FIG. 2 is a schematic cross-sectional view of the medical waste collection device of FIG. 1. FIG. 3 is a rear view of the medical waste collection device of FIG. 1. FIG. 4 is a bottom view of the medical waste collection device of FIG. 1. FIG. 5 is a schematic diagram of a pressing member of FIG. 2.

Referring to FIGS. 1 to 5, a medical waste collection device 10 according to an embodiment of the present invention may reduce the volume of medical waste to minimize the contact of medical workers with medical waste, thereby preventing the spread of contact infections. The medical waste collection device 10 may include a container 100, a cover 200, a compression member 300, a sterilization unit 400, a pedal 600, and wheels 700.

The container 100 may have a receiving space S therein to accommodate medical waste. The container 100 may have a first opening O1 on one side. In an embodiment, the first opening O1 may be located on the upper side of the container 100. The medical waste may be inserted into the receiving space S through the first opening O1.

The container 100 may hold a bag containing the medical waste therein. Accordingly, the bag may be placed in the receiving space S of the container 100. In an embodiment, the edge of the bag may protrude to the outside of the container 100.

The container 100 may be formed in a cylindrical shape. In an embodiment, the container 100 may be formed in a rectangular cylinder shape, but is not limited thereto. The container 100 may be made of synthetic resin, metal material, etc.

The container 100 may include a bottom portion and a side wall portion. The bottom portion may form the bottom of the container 100. In an embodiment, the bottom portion may be formed into a rectangular shape in a plan view. In contrast, in other embodiments, the bottom portion may be formed in various shapes, such as a circle, a triangle, etc.

The side wall portion may extend from the edge of the bottom portion toward the first opening O1. The side wall portion may form the perimeter of the container 100. The side wall portion may form at least one hole passing therethrough. In an embodiment, a transparent member (e.g., a glass window, etc.) may be positioned on the hole in the side wall portion. Accordingly, it is possible to visually check how much medical waste is piled up in the container 100 from the outside.

In contrast, in another embodiment, a pressure sensor installed on the side wall portion may measure the pressure applied by the medical waste to the side wall portion when the compression member 300 compresses the medical waste in the bag. When a pressure value measured by the pressure sensor is higher than a preset pressure value, the medical waste collection device 10 may notify the outside that the medical waste in the container 100 must be emptied, or may output the same through an output portion (not shown) installed in the medical waste collection device 10. The output portion may be an LED light source, a display, etc.

The cover 200 may be positioned on the container 100. The cover 200 may be removably installed on the container 100. Accordingly, the cover 200 may open and close the first opening O1. In an embodiment, the cover 200 may rotate on the container 100 to open and close the first opening O1.

The cover 200 may include an insertion hole 210 passing therethrough. A compression rod 320 of the compression member 300, which will be described later, may be located within the insertion hole 210. In an embodiment, the insertion hole 210 may be located in the central area of the cover 200. The cover 200 may include a cover plate 220, a cover peripheral portion 230, and curtain plates 240.

The cover plate 220 may be positioned above the first opening O1. The cover plate 220 may cover the first opening O1. In an embodiment, the cover plate 220 may be formed to be rectangular in a plan view.

The cover peripheral portion 230 may extend downward from the edge of the cover plate 220. Accordingly, the cover peripheral portion 230 may surround the upper side of the side wall portion of the container 100.

The curtain plates 240 may be installed on both sides of the cover peripheral portion 230. The curtain plates 240 may be inclined downward in a direction from the cover peripheral portion 230 to the container 100.

The compression member 300 may be installed on the cover 200. The compression member 300 may compress the medical waste contained in the bag to reduce the volume thereof. Accordingly, the compression member 300 may include a compression plate 310 and a compression rod 320.

The compression plate 310 may be positioned within the container 100. The compression plate 310 may be lifted and lowered within the container 100. The compression plate 310 may include a plurality of through holes 315 passing therethrough. Accordingly, the liquid absorbed in the medical waste may be discharged onto the compression plate 310 through the through holes 315, allowing the medical waste to be more effectively compressed. Additionally, ultraviolet rays from the sterilization unit 400 may reach the medical waste through the through holes 315 to sterilize the medical waste. In an embodiment, the through holes 315 may be arranged in a grid pattern, but are not limited thereto.

The compression rod 320 may be connected to the compression plate 310. In an embodiment, one end of the compression rod 320 may be installed in the middle area of the compression plate 310. The compression rod 320 may be detachably installed on the compression plate 310. The compression rod 320 may be formed as a cylindrical rod, but is not limited thereto. The end of the compression rod 320 may include a gripping portion.

The compression rod 320 may be inserted into the insertion hole 210 of the cover 200. The compression rod 320 may move up and down while passing through the cover 200. The compression rod 320 may press the compression plate 310. Accordingly, the compression plate 310 may compress the medical waste in the container 100 by the pressure applied by the compression rod 320.

The sterilization unit 400 may be installed on the container 100 or the cover 200. In an embodiment, the sterilization unit 400 may be installed on the lower part of the cover 200 so as to be positioned between the cover 200 and the compression plate 310. Accordingly, when the compression plate 310 is lifted or lowered within the container 100, interference with the sterilization unit 400 may be minimized.

The sterilization unit 400 may sterilize the medical waste contained in the container 100. The sterilization unit 400 may sterilize the medical waste in the bag mounted on the container 100. The sterilization unit 400 may include at least one light source portion for irradiating the medical waste in the container 100 with ultraviolet rays. Accordingly, bacteria, viruses, and the like in the medical waste may be sterilized. In an embodiment, the sterilization unit 400 may include a plurality of light source portions.

The pedal 600 may be installed on the container 100. The pedal 600 may provide a rotational force to the cover 200 rotatably installed in the container 100. Accordingly, the cover 200 may open and close the first opening O1 by receiving the rotational force of the pedal 600. The pedal 600 may include a first rotating member 610, a pedal member 620, an elevating member 630, and a second rotating member 640.

The first rotating member 610 may be elongated in one direction. The first rotating member 610 may be installed on the lower part of the container 100. In an embodiment, the first rotating member 610 may be rotatably installed on the lower part of the container 100. The first rotating member 610 may include a first hinge 613 and a first frame 611.

The first frame 611 may be elongated in one direction. The first frame 611 may include a first length adjustment hole 611a formed along its longitudinal direction. The first length adjustment hole 611a may pass through the first frame 611.

The first hinge 613 may be installed on the lower part of the container 100. The first hinge 613 may include a first rotating support and a second rotating support, each connected to the lower part of the container 100. The first frame 611 may be located between the first rotating support and the second rotating support. In an embodiment, the first rotating support and the second rotating support may be located approximately in the middle of the first frame 611 in the longitudinal direction, but are not limited thereto.

The first hinge 613 may include a first hinge shaft. The first hinge shaft may pass through the first rotating support, the second rotating support, and the first frame 611. Accordingly, the first frame 611 may rotate with respect to the first hinge shaft. The longitudinal direction of the first hinge shaft may be perpendicular to the longitudinal direction of the first frame 611.

The pedal member 620 may be installed on one side of the first rotating member 610. The pedal member 620 may receive the external force to rotate the first rotating member 610. The external force provided to the pedal member 620 may be the load of the user's foot. The pedal member 620 may include a pedal plate 621 and a connection frame 623.

The pedal plate 621 may be an area on which the user steps. Accordingly, the external force may be provided through the pedal plate 621. The pedal plate 621 may be positioned so as not to overlap the container 100. Accordingly, the user may easily step on the pedal plate 621.

The connection frame 623 may extend from the pedal plate 621 toward the first rotating member 610. The connection frame 623 may be elongated in one direction. The connection frame 623 may be positioned on the same line as the first frame 611. The connection frame 623 may include a second length adjustment hole 623a formed along its longitudinal direction. The second length adjustment hole 623a may penetrate the connection plate. At least a portion of the second length adjustment hole 623a may overlap the first length adjustment hole 611a.

The connection frame 623 may be connected to the first frame 611 through a fastening member (e.g., bolt, nut, etc.). In an embodiment, the fastening member may connect the first frame 611 to the connection frame 623 by passing through the first length adjustment hole 611a and the second length adjustment hole 623a. The user may adjust the position of the pedal plate 621 by adjusting the length of the area where the first length adjustment hole 611a and the second length adjustment hole 623a overlap.

The elevating member 630 may be installed on the other side of the first rotating member 610. The elevating member 630 may be lifted and lowered by rotating the first rotating member 610. In an embodiment, the elevating member 630 may move in an upward direction by rotation of the first rotating member 610. The elevating member 630 may be included in a second frame 631 and a third frame 633.

The second frame 631 may be elongated in the vertical direction. The second frame 631 may be configured to have an adjustable length. One side of the second frame 631 may be connected to one side of the first rotating member 610. The second frame 631 may include a fastening member connecting one side of the first frame 611 and one side of the second frame 631.

The third frame 633 may be installed in the container 100. In an embodiment, the third frame 633 may be installed on the side wall portion of the container 100. The third frame 633 may be elongated in the longitudinal direction of the second frame 631. The third frame 633 may include a bottom plate positioned on the container 100, and a first side wall plate and a second side wall plate, respectively extending from both sides of the bottom plate in a direction away from the container 100. The second frame 631 may be positioned between the first side wall plate and the second side wall plate.

The second rotating member 640 may be installed on one side of the cover 200. The second rotating member 640 may be connected to the other side of the elevating member 630. The second rotating member 640 may rotate the cover 200 by lifting the elevating member 630. In an embodiment, the second rotating member 640 may include a fourth frame 641 and a second hinge 643.

The fourth frame 641 may be installed on the cover 200. The fourth frame 641 may be elongated in one direction. In an embodiment, the fourth frame 641 may penetrate the cover peripheral portion 230 and may be installed on the lower part of the cover plate 220. A portion of the fourth frame 641 may be located outside the cover 200 and connected to the elevating member 630.

The second hinge 643 may be installed on one side of the fourth frame 641. The fourth frame 641 may rotate with respect to the second hinge 643 when the elevating member 630 moves upward. Accordingly, the cover 200 coupled to the fourth frame 641 may also rotate to open the first opening O1. The second hinge 643 may include a second hinge shaft elongated in a direction perpendicular to the longitudinal direction of the fourth frame 641. The second hinge shaft may pass through the fourth frame 641. The other side of the elevating member 630 may be positioned between the second hinge shaft and the other side of the fourth frame 641.

The wheels 700 may be installed at the lower part of the container 100. The wheels 700 may enable the container 100 to move. Accordingly, the user may easily move the medical waste collection device 10.

The user may minimize the volume of medical waste by compressing the medical waste contained in the bag mounted in the container 100 using the compression member 300. Additionally, the sterilization unit 400 may sterilize bacteria, viruses, etc. in the medical waste by irradiating the medical waste with ultraviolet rays, etc. Accordingly, the user may safely dispose of the medical waste from the medical waste collection device 10.

The user may use his or her foot to operate the cover 200 to open the first opening O1. Accordingly, the user may easily put the medical waste into the container 100 and dispose of the bag containing the medical waste out of the container 100.

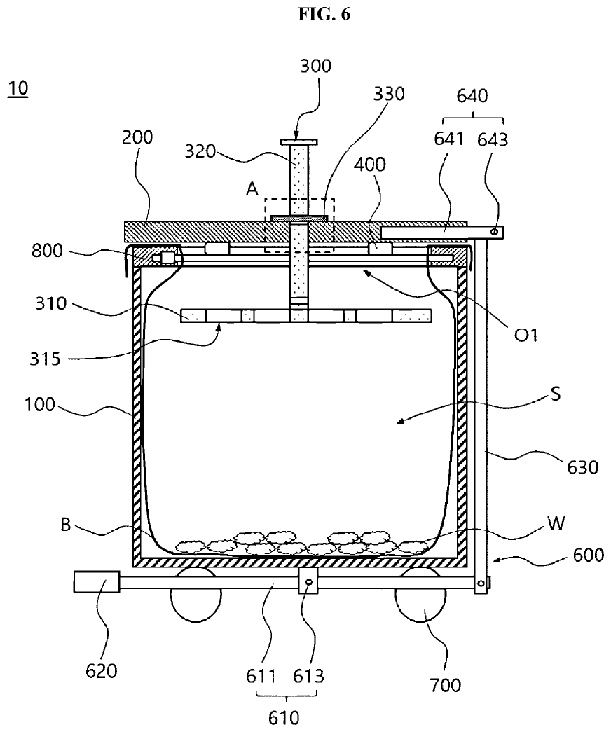

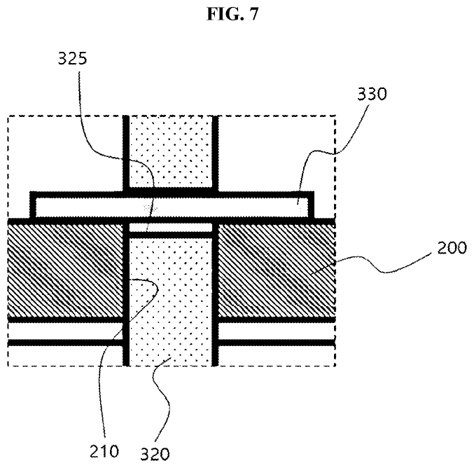

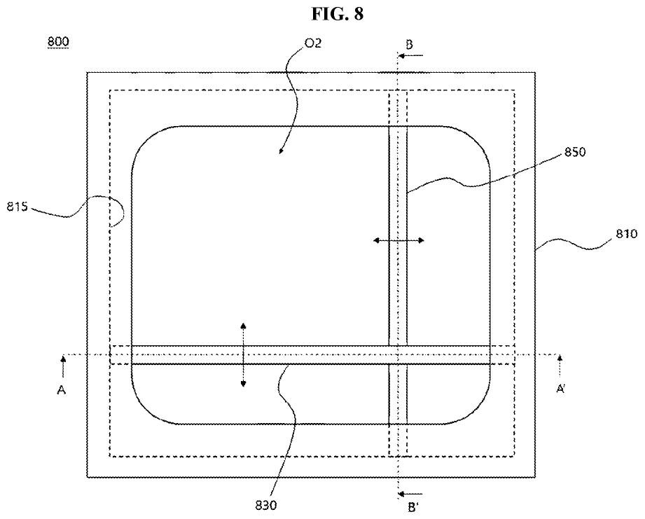

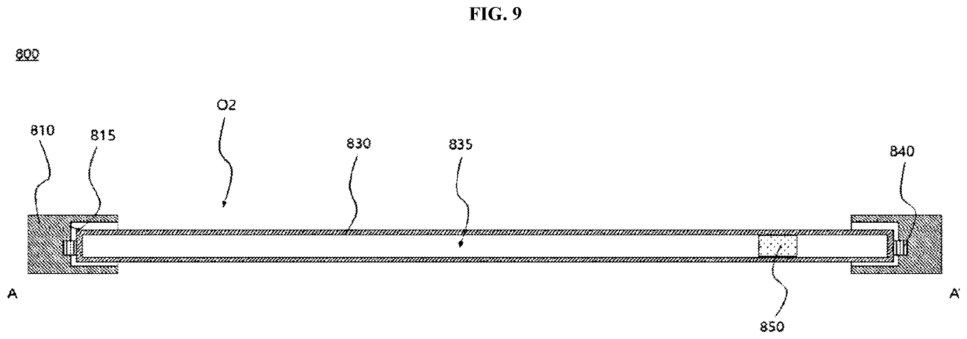

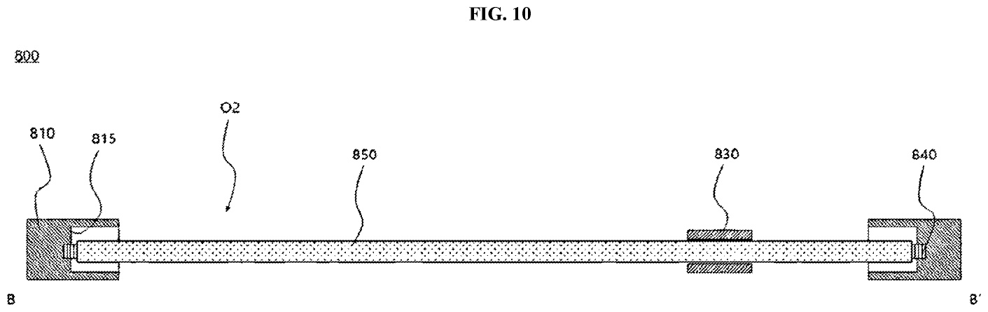

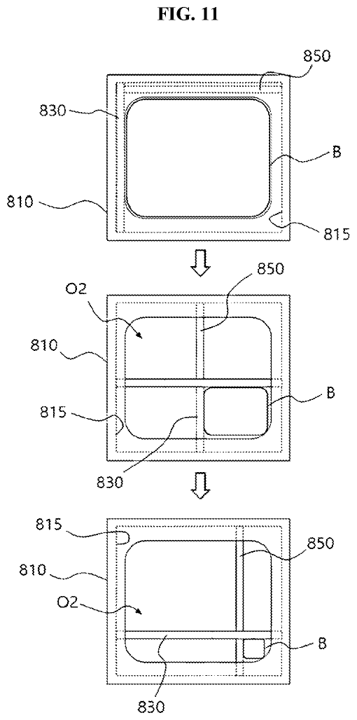

FIG. 6 is a schematic cross-sectional view of a medical waste collection device according to another embodiment of the present invention. FIG. 7 is an enlarged view of area A of FIG. 6. FIG. 8 is a plan view of a gripping unit of FIG. 6. FIG. 9 is a cross-sectional view taken along line A-A′ in FIG. 8. FIG. 10 is a cross-sectional view taken along line B-B′ in FIG. 8. FIG. 11 is a schematic diagram for illustrating a process in which the gripping unit of FIG. 8 grips an upper part of a bag. For convenience of explanation, the description of the same configuration as that described in FIGS. 1 to 5 will be omitted or briefly described.

Referring to FIGS. 6 to 11, a medical waste collection device 10 according to another embodiment of the present invention may include a container 100, a cover 200, a compression member 300, a sterilization unit 400, a pedal 600, and wheels 700. Additionally, the medical waste collection device 10 may further include a gripping unit 800.

The gripping unit 800 may be installed on the container 100. In an embodiment, the gripping unit 800 may be positioned between the cover 200 and the container 100. The gripping unit 800 may be detachably mounted on the container 100. The gripping unit 800 may close and grip an upper part of a bag B mounted on the container 100. Accordingly, the opening of the bag B at the top of the bag B containing medical waste W may be narrowed or may be sealed. The gripping unit 800 may include a body 810, a first gripping bar 830, a second gripping bar 850, and a driving unit 840.

The compression member 300 may include a compression plate 310 and a compression rod 320. The compression rod 320 may include at least one stopper hole 325 passing therethrough. In an embodiment, a plurality of stopper holes 325 may be provided. The plurality of stopper holes 325 may be arranged in the longitudinal direction of the compression rod 320.

The compression member 300 may further include a stopper member 330. The stopper member 330 may be located on the cover 200. The stopper member 330 may be inserted into any one of the stopper holes 325. Accordingly, the compression member 300 may be prevented from falling into the container 100 due to the load.

The body 810 may be located on the container 100. The body 810 may have a second opening O2 passing therethrough. The second opening O2 may be interlocked with the first opening O1 of the container 100. In an embodiment, the second opening O2 may overlap the first opening O1. The body 810 may be formed in a rectangular shape in a plan view.

The body 810 may include a movement groove 815 that is recessed from the inner surface surrounding the second opening O2 toward the outside. The movement groove 815 may be formed in a ring shape. The moving groove 815 may surround the second opening O2.

The first gripping bar 830 may be movably installed on the body 810. The first gripping bar 830 may move across the second opening O2. The first gripping bar 830 may be elongated in one direction. Both ends of the first gripping bar 830 may be positioned within the movement groove 815. The first gripping bar 830 may include a guide hole 835 passing therethrough. The guide hole 835 may be elongated in the longitudinal direction of the first gripping bar 830. The first gripping bar 830 may be moved along the second gripping bar 850 by the driving unit 840.

The second gripping bar 850 may be elongated in one direction. The second gripping bar 850 may intersect with the first gripping bar 830 perpendicularly. The second gripping bar 850 may move across the second opening O2. The second gripping bar 850 may pass through the guide hole 835 of the first gripping bar 830. Accordingly, the second gripping bar 850 may be inserted into the guide hole 835. The second gripping bar 850 may move within the guide hole 835. The second gripping bar 850 may be moved along the first gripping bar 830 by the driving unit 840.

The driving unit 840 may move the first gripping bar 830 and the second gripping bar 850. The driving unit 840 may be installed in the body 810. In an embodiment, the driving unit 840 may be installed in the movement groove 815. The driving unit 840 may be connected to both ends of the first gripping bar 830 and both ends of the second gripping bar 850. Accordingly, the driving unit 840 may provide the driving force to the first gripping bar 830 and the second gripping bar 850.

The upper part of the bag B may be located between the body 810 and the cover 200. The bag B may be inserted into the container 100 through the second opening O2 and the first opening O1. The cover 200 may open and close the second opening O2 together with the first opening O1.

The driving unit 840 may move the first gripping bar 830 and the second gripping bar 850 across the second opening O2. The first gripping bar 830 and the second gripping bar 850, which move across the second opening O2, may close the upper part of the bag B passing through the second opening O2 toward the edge of the second opening O2. Before the first gripping bar 830 and the second gripping bar 850 pull the upper part of the bag B toward the edge of the second opening O2, it is desirable to position the compression plate 310 adjacent to the lower part of the cover 200. When the compression plate 310 is positioned adjacent to the lower part of the cover 200, it is possible to prevent the movement of the first gripping bar 830 and the second gripping bar 850 from being restricted due to interference of the compression plate 310.

The first gripping bar 830 and the second gripping bar 850 may grip the upper part of the bag B while pulling the upper part of the bag B toward the edge of the second opening O2. At this time, the user may rotate the cover 200 to open the first opening O1 and the second opening O2. The user may tie the upper part of the bag B closed and gripped by the first gripping bar 830 and the second gripping bar 850. Accordingly, the user may seal the bag B.

In another embodiment, the first gripping bar 830 and the second gripping bar 850 may each include a heat fusion unit that seals the bag B by heat fusion of the bag B. The heat fusion unit may be positioned at one end of each of the first gripping bar 830 and the second gripping bar 850.

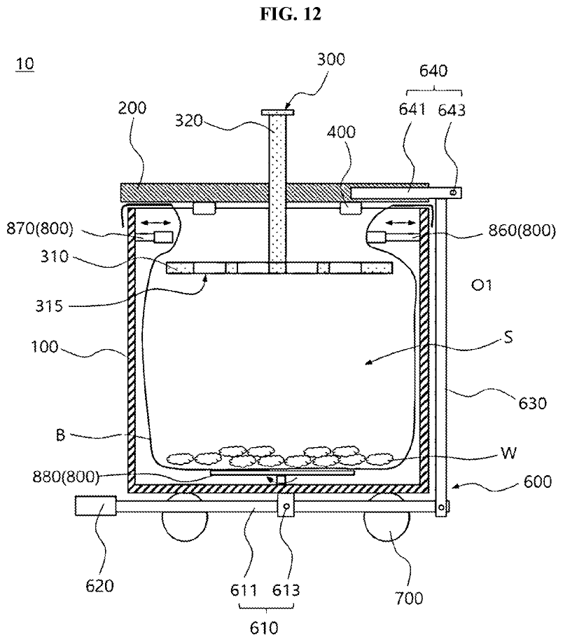

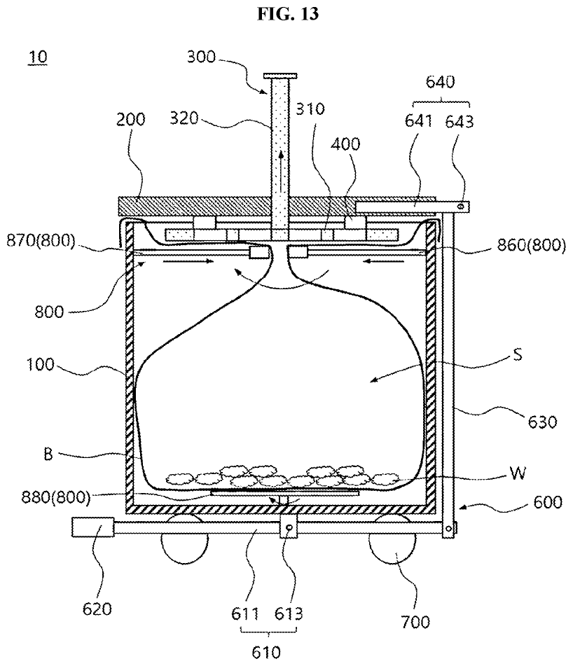

FIG. 12 is a schematic cross-sectional view of a medical waste collection device according to another embodiment of the present invention. FIG. 13 is a schematic diagram of the medical waste collection device of FIG. 12 sealing a bag. For convenience of explanation, the description of the same configuration as that described in FIGS. 1 to 5 will be omitted or briefly described.

Referring to FIGS. 11 and 12, a medical waste collection device 10 according to another embodiment of the present invention may include a container 100, a cover 200, a compression member 300, a sterilization unit 400, a pedal 600, and wheels 700. Additionally, the medical waste collection device 10 may further include a gripping unit 800.

The gripping unit 800 may be installed on the container 100. The gripping unit 800 may include a first gripping portion 860, a second gripping portion 870, and a rotating portion 880. The first gripping portion 860 and the second gripping portion 870 may be installed in the container 100. In an embodiment, the first gripping portion 860 and the second gripping portion 870 may be spaced apart from each other. The first gripping portion 860 may face the second gripping portion 870.

The first gripping portion 860 and the second gripping portion 870 may be located adjacent to the first opening O1. Each of the first gripping portion 860 and the second gripping portion 870 may move in the inner and outer directions of the container 100. The first gripping portion 860 and the second gripping portion 870 may move in the inner direction of the container 100 to become closer to each other. Accordingly, the first gripping portion 860 and the second gripping portion 870 may pull the upper part of the bag B toward the middle area of the container 100. Additionally, the first gripping portion 860 and the second gripping portion 870 may grip the upper part of the bag B.

The rotating portion 880 may be installed within the container 100. The rotating portion 880 may be installed on the bottom of the container 100. The rotating portion 880 may rotate within the container 100. The bag B may be placed on the rotating portion 880. Accordingly, as the rotating portion 880 rotates, the bag B may rotate.

The gripping unit 800 may rotate the rotating portion 880 while the first gripping portion 860 and the second gripping portion 870 grip the upper part of the bag B. As the bag B is rotated by the rotating portion 880, the upper part of the bag B gripped by the first gripping portion 860 and the second gripping portion 870 may be twisted. By twisting the upper part of the bag B, the bag B may be sealed.

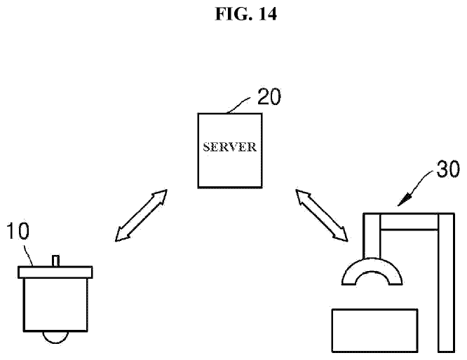

FIG. 14 is a schematic diagram of a medical waste collection system including a medical waste collection device according to an embodiment of the present invention.

Referring to FIG. 14, the medical waste collection device 10 according to an embodiment of the present invention may be used in a medical waste collection system. The medical waste collection system may include a server 20, a medical waste collection device 10, and a medical waste pickup device 30.

The medical waste collection device 10 may be the medical waste collection device 10 described above with reference to FIGS. 1 to 13. Additionally, the medical waste collection device 10 may further include a communication unit (not shown) that communicates wirelessly. When the bag contained in the container 100 (see FIG. 1) is filled with medical waste, the medical waste collection device 10 may seal the bag. When sealing the bag, the medical waste collection device 10 may transmit sealing information of the bag, information on the bag filled with medical waste, and the like to the server 20 through the communication unit.

The server 20 may transmit a movement signal to the medical waste collection device 10 so that the medical waste collection device 10 moves to the medical waste pickup device 30 when receiving the sealing information and the like from the communication unit.

When receiving the movement signal, the medical waste collection device 10 may move to the medical waste pickup device 30 according to movement path information, location information of the medical waste pickup device 30, and the like included in the movement signal.

In an embodiment, the medical waste collection device 10 may autonomously move to the medical waste pickup device 30. Alternatively, in another embodiment, the medical waste collection device 10 may be moved by the user.

When the medical waste collection device 10 moves to the location of the medical waste pickup device 30, the medical waste collection device 10 may transmit information that movement to the location of the medical waste pickup device 30 has been completed to the server 20 through the communication unit.

When the server 20 receives a signal that the movement has been completed from the medical waste collection device 10, the server 20 may send pickup information to the medical waste pickup device 30 so that the medical waste pickup device 30 picks up the bag in the medical waste collection device 10.

When the medical waste pickup device 30 receives the pickup information from the server 20, the medical waste pickup device 30 may pick up the bag in the container of the medical waste collection device 10. The medical waste pickup device 30 may pick up the bag and then place the bag into a box member to receive the bag. When the bag is placed within the box member, the medical waste pickup device 30 may seal the box member with tape or the like. The medical waste pickup device 30 may move the sealed box member to a collection location.

Alternatively, in another embodiment, the box member may be located within the container of the medical waste collection device 10. Additionally, the bag may be placed within the box member. The medical waste collection device 10 may seal the box member when the bag is sealed using a separate device, or the user may seal the box member.

Although the preferred embodiments of the present invention have been particularly shown and described, the present invention is not limited to the embodiments including the features described above. In addition, it will be understood by a person skilled in the art that various changes in form and details may be made therein without departing from the spirit and scope of the following claims.

1. A medical waste collection device comprising:
a container having a first opening on a side and configured for holding a bag therein;
a cover detachably installed in the container and configured to open and close the first opening;
a compression member installed in the cover and configured for compressing medical waste contained in the container, the compression member comprising a compression plate located within the container, and a compression rod passing through the cover and connected to the compression plate, the compression rod configured to press the compression plate; and
a sterilization unit configured for sterilizing the medical waste contained in the container.

2. The medical waste collection device of claim 1, wherein the compression plate comprises a plurality of through holes passing therethrough.

3. The medical waste collection device of claim 2, wherein the sterilization unit comprises at least one light source portion for irradiating the medical waste contained in the container with ultraviolet rays, and
the light source portion is installed in the cover so as to be positioned between the compression plate and the cover.

4. The medical waste collection device of claim 1, further comprising a pedal installed in the container and providing the rotational force to the cover rotatably installed in the container,
wherein the pedal comprises:
a first rotating member elongated in one direction and rotatably installed at a lower part of the container;
a pedal member installed on one side of the first rotating member and configured for rotating the first rotating member by receiving the external force;
an elevating member installed on the other side of the first rotating member, the elevating member configured to be lifted and lowered by rotation of the first rotating member; and
a second rotating member installed on one side of the cover, the second rotating member configured to rotate the cover through the lifting and lowering of the elevating member.

5. The medical waste collection device of claim 1, further comprising wheels installed at the lower part of the container and configured for enabling the container to move.

6. The medical waste collection device of claim 1, further comprising a gripping unit installed on the container and configured for closing and gripping the upper part of the bag mounted on the container.

7. The medical waste collection device of claim 6, wherein the gripping unit comprises:
a body having a second opening in communication with the first opening;
a first gripping bar installed on the body and elongated in one direction;
a second gripping bar that is elongated in one direction and intersects with the first gripping bar perpendicularly; and
a driving unit configured to move the first gripping bar and the second gripping bar;
wherein the first gripping bar is configured to be moved along the second gripping bar by the driving unit while crossing the second opening, and
the second gripping bar is configured to be moved along the first gripping bar by the drive unit while crossing the second opening.

8. The medical waste collection device of claim 7, wherein the first gripping bar comprises a guide hole passing therethrough and elongated in the longitudinal direction, and
the second gripping bar is inserted into the guide hole and is configured to be moved within the guide hole.

9. A medical waste collection system comprising:
the medical waste collection device of claim 1;
a medical waste pickup device configured to pick up the bag in the container of the medical waste collection device; and
a server configured to wirelessly communicate with the medical waste collection device and the medical waste pickup device and control the medical waste collection device and the medical waste pickup device;
wherein, when the bag in the container is sealed, the medical waste collection device is configured to transmit sealing information to the server through a communication unit, and
when the sealing information is received, the server is configured to transmit a movement signal to cause the medical waste collection device to move to the medical waste pickup device.
